(19) United States
(12) Patent Application Publication
(10) Pub. No.: US 2024/0324959 A1
(43) Pub. Date: Oct. 3, 2024

(54) METHOD AND SYSTEM FOR VALIDATING OPTIMAL BODY SENSOR PLACEMENT

(71) Applicant: KONINKLIJKE PHILIPS N.V., Eindhoven (NL)

(72) Inventors: Boris Emmanuel Rachmund De Ruyter, Eindhoven (NL); Joanne Henriette Desiree Monique Westerink, Eindhoven (NL)

(21) Appl. No.: 18/619,455

(22) Filed: Mar. 28, 2024

Related U.S. Application Data
(60) Provisional application No. 63/456,056, filed on Mar. 31, 2023.

(51) Int. Cl. A61B 5/00; A61B 5/0205; A61B 5/024; A61B 5/0533; A61B 5/259; A61B 5/318

(52) U.S. Cl. CPC A61B 5/6833; A61B 5/0002; A61B 5/0205; A61B 5/0533; A61B 5/443; A61B 5/742; A61B 5/02438; A61B 5/259; A61B 5/318; A61B 2562/0215

(57) ABSTRACT

Disclosed is a system comprising a monitoring patch configured to be adhesively attached to skin of an associated patient, the monitoring patch including at least one vital sign sensor arranged to measure vital sign data of the associated patient when the monitoring patch is adhesively attached to the skin of the associated patient. The monitoring patch includes a wireless transceiver or transmitter configured to wirelessly transmit the vital sign data, a monitoring device configured to receive the wirelessly transmitted vital sign data and to store, retransmit, or both store and retransmit the vital sign data, a skin condition sensor configured to measure skin measurement data on the skin of the associated patient, and at least one hardware processor configured to analyze the skin measurement data to determine advice on adhesive attachment of the monitoring patch to the skin and output the advice on the monitoring patch or the monitoring device.

### CROSS-REFERENCE TO RELATED APPLICATIONS

This patent application claims the priority benefit under 35 U.S.C. § 119 (c) of U.S. Provisional Application No. 63/456,056, filed on Mar. 31, 2023, the contents of which are herein incorporated by reference.

The following relates generally to the medical monitoring arts, wearable medical monitor arts, heart rate monitoring arts, patient activity monitoring arts, and related arts.

### BACKGROUND

Health-related unobtrusive sensing systems enable replacement of continued hospitalization with obtrusive vital signs sensor technologies, centered around the individual, to provide remote monitoring of the subject's general health condition. Such systems can also be used for other purposes such as diagnosis of cardiac conditions, screening for medical conditions, and so forth. Vital signs monitoring typically includes monitoring one or more of the following physical parameters: heart rate (HR), blood pressure (BP), respiratory rate (RR), core body temperature and blood oxygenation (SpO₂).

Current ambulatory ECG monitors and mobile cardiac telemetry systems gather ECG data in ambulatory settings (e.g. in the patient's home environment or on-the-go). In such systems, a patient places the sensor as a patch on the chest. The sensor communicates wirelessly with a dedicated mobile device, for example similar in form factor to a smartphone, which is connected via the Internet to the clinical service center. In other embodiments, the sensor may be directly connected to the Internet, or may utilize an application program (“app”) running on a general purpose smartphone. Some examples of such devices include the ePatch (Extended Holter Monitor) and MCOT (Mobile Cardiac Telemetry) devices available from Philips BioTel. These devices provide a wearable single-use electrodes patch onto which an electronics module attaches to form a patient-worn device. The electronics module is advantageously reusable, at least for a given patient.

With the current advances in wearable technologies and connected platforms, remote patient monitoring has become the preferred approach for collecting in-situ data for both diagnosis and treatment. An important aspect in remote patient monitoring is the installation and activation of sensing technologies to capture data. Typically, such installation and activation is performed by the patients themselves. Adequate guidance in this process is critical for the quality of the remote patient monitoring system.

Many wearable sensors obtain their measurements via contact with the skin. In this case, the patient should be guided in installing the sensor on the proper place on the body, as well as in preparing the skin of that part of the body, such that the contact is optimal. Certain properties of the skin, like sebum level (i.e., greasiness) or hair density, prevent proper attachment of a wearable to the skin. The patient should prepare the skin such that adhesion is optimal. Afterwards, the patient needs to attach the wearable to the skin, in such a way that the skin contact of the relevant sensors is sufficient and optimal.

In most cases, patients are not familiar with the installation and activation of remote monitoring technologies. Additionally, due to their healthcare condition, patients tend to be stressed when having to perform this process.

The following discloses certain improvements to overcome these problems and others.

### SUMMARY

In one aspect, a system includes a monitoring patch configured to be adhesively attached to skin of an associated patient. The monitoring patch includes at least one vital sign sensor arranged to measure vital sign data of the associated patient when the monitoring patch is adhesively attached to the skin of the associated patient. The monitoring patch further includes a wireless transceiver or transmitter configured to wirelessly transmit the vital sign data. A monitoring device is configured to receive the wirelessly transmitted vital sign data and to store, retransmit, or both store and retransmit the vital sign data. A skin condition sensor is configured to measure skin measurement data on the skin of the associated patient. At least one hardware processor is configured to analyze the skin measurement data to determine advice on adhesive attachment of the monitoring patch to the skin; and output the advice on the monitoring patch or the monitoring device.

In another aspect, a health status monitoring method includes measuring, with a skin condition sensor attached to skin of an associated patient, skin measurement data on the skin of the associated patient; analyzing, with at least one hardware processor, the skin measurement data to determine advice on adhesive attachment of a monitoring patch to the skin; and outputting the advice.

One advantage resides in providing a reliable remote monitoring system for monitoring a patient.

Another advantage resides in ensuring adherence of a patient monitoring system to the skin of a patient.

Another advantage resides in an easy-to-install wearable patient monitoring device, thereby reducing patient stress.

Another advantage resides in determining a status of skin of a patient for adhering a wearable patient monitoring system to the skin of the patient,

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIGS. 1, 2, and 3 diagrammatically shows various illustrative embodiments of a patient monitoring system in accordance with the present disclosure.

FIGS. 4 and 5 show example flow charts of operations suitably performed by the system of FIG. 1.

### DETAILED DESCRIPTION

As used herein, the singular form of “a,” “an”, and “the” include plural references unless the context clearly dictates otherwise. As used herein, statements that two or more parts or components are “coupled,” “connected,” or “engaged” shall mean that the parts are joined, operate, or co-act together either directly or indirectly, i.e., through one or more intermediate parts or components, so long as a link occurs. Directional phrases used herein, such as, for example and without limitation, top, bottom, left, right, upper, lower, front, back, and derivatives thereof, relate to the orientation of the elements shown in the drawings and are not limiting upon the scope of the claimed invention unless expressly recited therein. The word “comprising” or “including” does not exclude the presence of elements or steps other than those described herein and/or listed in a claim. In a device comprised of several means, several of these means may be embodied by one and the same item of hardware.

Disclosed systems focus on the placement of body-worn sensors (referred to herein as a patch) which requires thorough preparation such as the removal of body hair and the cleaning of the skin (i.e., to remove any lotions and oils from the skin). Failing to perform proper skin preparation will have a significant impact on both the quality of the patch placement (as it will not be firmly attached to the body) and the quality of the data collection. For patients, it is however not clear whether the preparation is adequate for achieving the desired quality for the remote monitoring system. The disclosed system assesses, through a sensor, the skin preparation quality during patch installation and provide direct feedback to the patient if additional preparation measures are required. Additionally, some embodiments of the disclosed system can be used to monitor if the patch is well attached over time.

Some illustrative embodiments employ a galvanic skin response (GSR) sensor, which has electrodes (e.g., Ag/AgCl electrodes, which can comprise an adhesive material) that contact the skin, and a low voltage or electric current source to apply a voltage or current across the electrodes to measure the electrical resistance (or conductance, which is the inverse of resistance) of the skin between the electrodes. GSR sensors are commonly used to assess emotional state (e.g., stress), as certain emotions trigger the autonomic nervous system to increase sweat emission, which in turn increases the electrical conductance of the skin (i.e. reduces the electrical resistance of the skin). The GSR sensor measurements can thus provide biofeedback for monitoring emotional state, for example to indicate when the user is experiencing a high level of stress. GSR sensors are sometimes incorporated into a smartwatch to provide the wearer with real-time emotional state feedback.

As described herein, a GSR sensor is used for a different purpose, namely, to assess whether skin has been properly prepared for placement of a heart rate monitoring device or the like. The GSR sensor measurement is performed immediately after preparing the skin. In one suitable skin preparation sequence, the skin is thoroughly washed with soap and then dried, followed by further scrubbing of the skin using a scrub pad. The GSR sensor measurement performed immediately after the skin preparation thus provides a quantitative assessment of whether the skin preparation has been sufficient to remove any sweat, oils, sebum, or the like to ensure a good electrical contact between the prepared skin and the heart rate monitor.

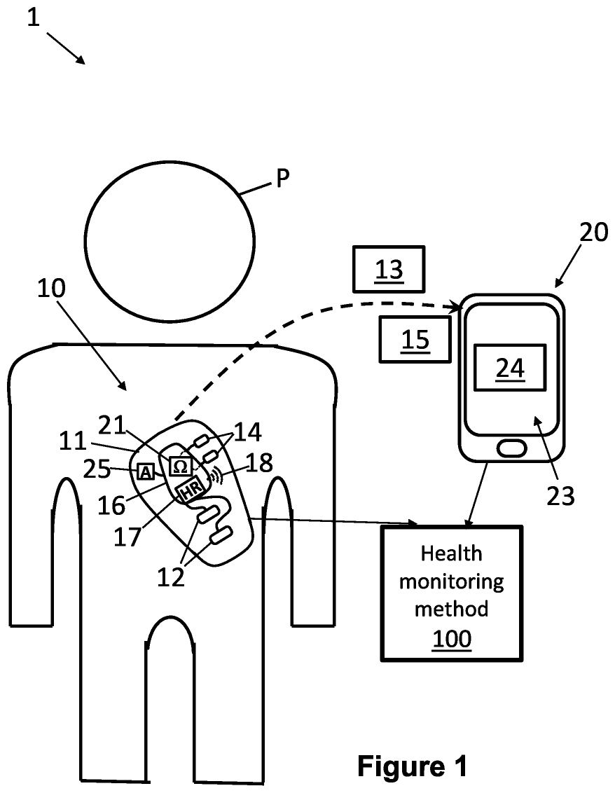

With reference to FIG. 1, a system 1 for monitoring an associated patient P is shown. As used herein, the term “patient” (and variants thereof) refers to, and includes, an outpatient, a discharged patient, a patient undergoing screening using the monitoring device as part of an annual medical checkup, or other person whose health condition is to be monitored. As shown in FIG. 1, the system 1 includes a wearable monitoring device 10 that is wearable by, or otherwise attached to (i.e., adhesively attached to), the patient P. The wearable monitoring device 10 can include any suitable monitoring device, such as a Mobile Cardiac Outpatient Telemetry (MCOT)® device (available from Philips ECG Solutions, Malvern, Pennsylvania, USA), or a medical wearable device, a torso-worn vital signs health patch, a wrist-worn watches, a chest strap, a smart garment, medical ear buds/over the Ear, a forehead or nose sensor, a smart ring, or so forth.

The wearable monitoring device 10 more generally can include one or more sensors configured to measure physiological sensor data of the patient P wearing the monitoring device 10. As shown in FIG. 1, the monitoring device 10 includes two sensors-a vital signs sensor 12 (referred to herein as an ECG sensor 12) and a galvanic skin response (GSR) sensor 14. The ECG sensor 12 is configured (e.g., comprising skin-contacting electrodes with silver/silver chloride coatings, for example) to measure cardiac data 13 of the patient P. The GSR sensor 14 is configured (e.g., comprising skin-contacting electrodes with silver/silver chloride coatings, for example) to measure a condition of the skin of the patient P on which the monitoring device 10 is disposed.

The illustrative wearable monitoring device 10 includes a single-use electrode patch 11 with one or more vital sign sensors 12 (e.g., which can be embodied as one or more electrodes 12) configured to measure vital sign data 13 of the patient P when the monitoring device 10 is adhesively attached to the skin of the patient P. As primarily described herein, the vital sign sensor(s) 12 comprise an electrocardiogram or heart rate monitor configured to measure cardiac data 13; although other types of vital sign sensors can be implemented (e.g., blood pressure sensors, pulmonary sensors, SpO₂ sensors, electroencephalogram (EEG) sensors, and so forth). Similarly, the GSR sensor 14 (e.g., embodied as electrodes 14) is configured to measure skin measurement data 15 on the skin of the patient P. The electrodes 14 can be integrated with the monitoring device 10 in the same patch 11 and arranged to electrically contact the skin when the monitoring device 10 is adhesively attached to the skin of the patient P.

The illustrative monitoring device 10 includes an electronics module 16 that attaches onto the electrodes patch 11, and the vital sign sensor 12 may be integrated into the electronics module 16. In illustrative FIG. 1, the electronics module 16 includes a heart rate (HR) module 17, for example comprising sample-and-hold and analog-to-digital conversion (ADC) circuitry for acquiring digital HR samples. The electronics module 16 further includes at least one microprocessor or microchip (not shown) configured (e.g. programmed) to optionally preprocess the cardiac signals from the HR module 17 from the cardiac sensor (electrodes) 12 to produce the cardiac data 13. The device 10 including the electrodes patch 11 and the attached electronics module 16 is adhesively secured to the chest or other anatomy of the patient P after suitable cleaning of the skin. The use of the separate electrodes patch 11 and electronics module 16 advantageously enables the single-use electrodes patch 11 to be a low-cost component that can be replaced as needed over the course of a patient monitoring session (which may extend over multiple days or weeks) while re-using the more expensive electronics module 16. However, other arrangements are contemplated such as having the patch 11 and electronics 16 constructed as a unitary single-use unit. Although not shown, it will be appreciated that the wearable monitoring device 10 also includes an on-board battery or other on-board electrical power source to power the electronics module 16. The on-board battery may, for example, be integrated with the electronics module 16, which may include a recharging port connector for recharging the electronics module 16, or the electronics module 16 could be placed on a wireless inductive recharging station to recharge it if needed during a patient monitoring session.

The wearable monitoring device 10 also includes a diagrammatically indicated wireless transmitter or transceiver 18 (referred to hereinafter as a transceiver 18), which may optionally be integrated with the electronics module 16. The transceiver 18 is integrated with or in wireless communication with the monitoring device 10 to transmit the patient data (e.g. the cardiac data 13 and skin measurement data 15) to a monitoring device 20 configured to receive the wirelessly transmitted vital sign data 13 and to store, retransmit, or both store and retransmit the vital sign data 13. As shown in FIG. 1, the monitoring device 20 comprises a mobile device 20 operable by the patient P or a clinician monitoring the patient P. In some embodiments, the electronic processor 16 is configured to collect and optionally preprocess the cardiac data 13 from the ECG sensor 12 and/or the skin measurement 15 from the GSR sensor 14 and a GSR module 21 also included in the electronics module 16, and the transceiver 18 is configured to transfer the cardiac data 13 and/or the skin measurement data 15 to the mobile device 20 (e.g., a cellphone or other smart device, or a dedicated medical monitoring device) operable by the patient P. In a typical arrangement, the transceiver 18 is a low-power wireless transceiver (e.g., Bluetooth™, Zigbee™, or the like) that connects with low power to the mobile device 20, thus placing a low power draw on the on-board battery of the wireless monitoring device 10.

Using the intermediary mobile device 20 has certain advantages such as providing a display 23 on which a user interface (UI) 24 can be displayed, e.g. to present a diary for the patient to record health symptoms. The mobile device 20 can also perform some or all processing of the cardiac and accelerometer data 13 and 15 instead of performing that processing at the on-board electronics module 16 of the wearable monitoring device 10. However, it is alternatively contemplated to omit the separate mobile device 20 and instead have all processing performed by the electronics module 16 of the wearable monitoring device 10, and to have the transceiver 18 of the wearable monitoring device 10 wirelessly communicate directly with the clinical health information system 22. In such embodiments the wearable monitoring device may also include a display for presenting the UI 24—for example, the wearable monitoring device 10 could have the form factor of a wristwatch.

In some embodiments, as shown in FIG. 1, the monitoring device 10 includes the vital sign sensor 12, the electronic processor (i.e., an on-board electronic processor) 16, and the wireless transceiver 18 are disposed on the patch 11 attachable to a portion of the patient P, and the GSR sensor 14 is also included in the same patch, and connected with the electronics module 16 which is further programmed to perform the GSR measurement. To this end, the electronics module 16 includes the diagrammatically indicated GSR module 21 which may for example comprise an ohmmeter (52) configured to apply a voltage (V) to the GSR electrodes 14 and measure the electrical current (I) through the portion of the skin disposed between the GSR electrodes 14. The GSR measurement is then given by the ratio I/V. In an alternative approach, the GSR may be measured as a conductance value, i.e. I/V. Herein, GSR is to be understood as encompassing cither an electrical resistance (V/I) or conductance (I/V) measurement. Furthermore, it is contemplated for the GSR module 21 may instead apply the current (I) and measure the voltage (V). In either case, the on-board battery or other on-board electrical power source that powers the electronics module 16 also suitably powers the GSR module 21 to deliver the applied voltage (V) or current (I) to the GSR electrodes 14 to perform the GSR measurement.

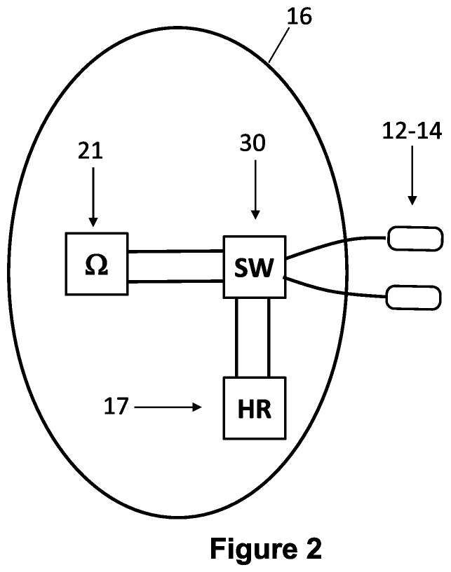

With continuing reference to FIG. 1 and with further reference to FIG. 2, in another embodiment the same electrodes are used for both the heart rate (or other vital sign) measurement and for the GSR measurement. In FIG. 2, an isolation view of such an alternative embodiment is shown, which illustrates only the electronics module 16 and the electrodes 12-14 which in this embodiment are used for both GSR and heart rate (HR) measurements. In this embodiment, the heart rate module 17 should not measure heart rate while the GSR module 21 is measuring GSR, because the voltage (or current) applied during the GSR measurement could interfere with an ECG measurement. Accordingly, as diagrammatically shown in FIG. 2, the electronics module 16 in this embodiment includes a switch 30 to switch between: (i) connecting the electrodes 12-14 with the heart rate monitor 17 (and not the GSR monitor 21), or (ii) connecting the electrodes 12-14 with the GSR monitor 21 (and not the heart rate monitor 17). The switch 30 could be implemented as a MOSFET switch or the like.

In the case of the embodiment of FIG. 1 which employs separate HR electrodes 12 and GSR electrodes 14, it may be feasible to measure both HR and GSR simultaneously, depending on factors such as how far apart the HR electrodes 12 are from the GSR electrodes 14 and the electrical power (voltage or current) applied during GSR measurement. If the GSR may adversely affect HR measurements then the processor of the electronics module 16 can be programmed to stop HR measurement during GSR measurements.

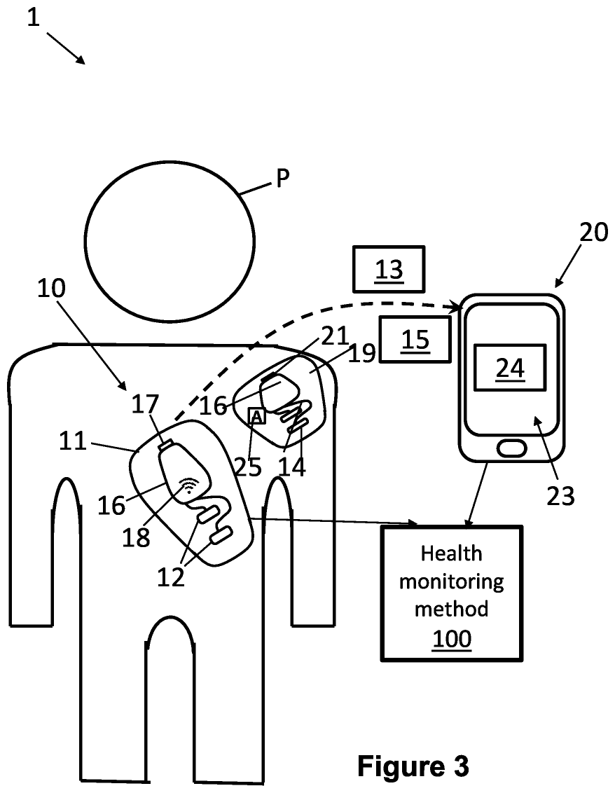

In some further embodiments, as shown in FIG. 3, the monitoring device 10 includes the vital sign sensor 12, the electronic processor (i.e., an on-board electronic processor) 16, and the wireless transceiver 18 are disposed on the patch 11 attachable to a first portion of the patient P, and the GSR sensor 14 is disposed on a second, separate patch 19 attachable to a second portion of the patient P. The second patch 19 also includes the GSR module (not indicated in FIG. 3) and an electrical source 21 connected to apply electrical current or voltage to the GSR electrodes 14 to measure the skin measurement data 15. In one embodiment, the on-board electronic processor 16 is disposed on the first patch 11, and is configured to control the vital sign sensor 12 to acquire the vital sign data 13 using the vital sign electrodes 12. In another embodiment, the on-board electronic processor 16 is disposed on the second patch 19, and is configured to control the GSR sensor 14 to acquire the skin measurement data 15 using the GSR electrodes 14. In another embodiment, the GSR electrodes 14 are the same set of electrodes as the vital sign electrodes 12. In this embodiment, the on-board hardware processor 16 is configured to control operation of the vital sign sensor 12 and the GSR sensor 14 to not measure the vital sign data 13 using the vital sign sensor 12 when the GSR sensor is operating to acquire the GSR skin measurement data 15.

Once acquired, the hardware processor 16 (or, alternatively, the monitoring device 20) is configured to analyze the skin measurement data 15 to determine advice on adhesive attachment of the monitoring patch 10 to the skin of the patient P, and output the advice on the monitoring patch 10 or the monitoring device 20. In some examples, the advice comprises a recommendation as to whether the skin is properly prepared for adhesive attachment of the monitoring patch 10 to the skin. In another example, the advice comprises an estimated remaining operating time of the monitoring patch 10 adhesively attached to the skin (i.e., due to loss of adhesion or degradation of the electrode contact with the skin).

In one example, the advice is output on the GUI 24 of the mobile device 20. In another example, the second patch 19 further includes a light emitting diode 25, and the hardware processor 16 is configured to operate the LED 25 in a first state if the skin is properly prepared and in a second state different from the first state if the skin is not properly prepared. For example, the LED 25 can be illuminated or not illuminated to show the patient P whether the skin is properly prepared or not. In another example, the LED 25 can be illuminated in a first color (i.e., green) if the skin is properly prepared and illuminated in a second color (i.e., red) if the skin is not properly prepared.

As further diagrammatically indicated in FIGS. 1 and 3, the electronic processor 16 is configured to perform a health status monitoring method 100 of monitoring the patient P by analyzing the cardiac data 13 and/or the skin measurement data 15.

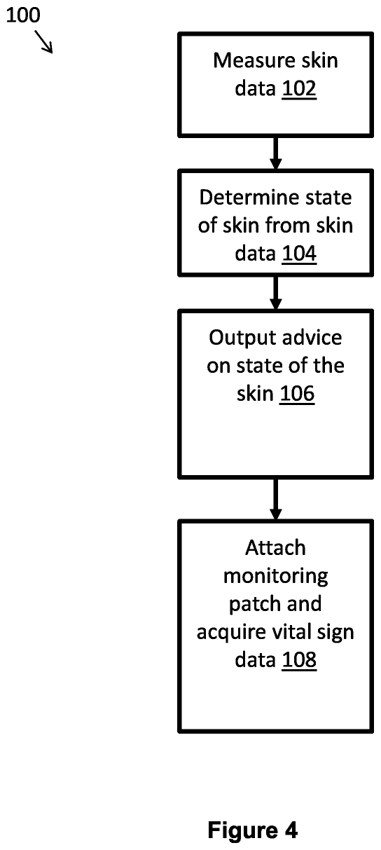

With reference now to FIG. 4, an illustrative embodiment of the health status monitoring method 100 is shown by way of a flowchart. To begin the method 100, the second patch 19 that includes the GSR sensor 14 is attached to the patient P. At an operation 102, the electronic processor 16 is configured to control the GSR sensor 14 to acquire the skin measurement data 15. At an operation 104, the electronic processor 16 is configured to analyze the skin measurement data 15 to determine advice on adhesive attachment of the monitoring patch 10 (i.e., the patch 11 containing the vital sign sensor 12) to the skin based on the skin measurement data 15. At an operation 106, the electronic processor 16 is configured to output the advice (e.g., on the GUI 24 of the mobile device 20, by controlling the LED 25, and so forth). At an operation 108, when the advice is indicative of the skin being properly prepared for adhesive attachment of the monitoring patch 10 to the skin, then the patch 11 is attached to the skin of the patient P, and the vital sign sensor 12 is configured to acquire the vital sign data 13.

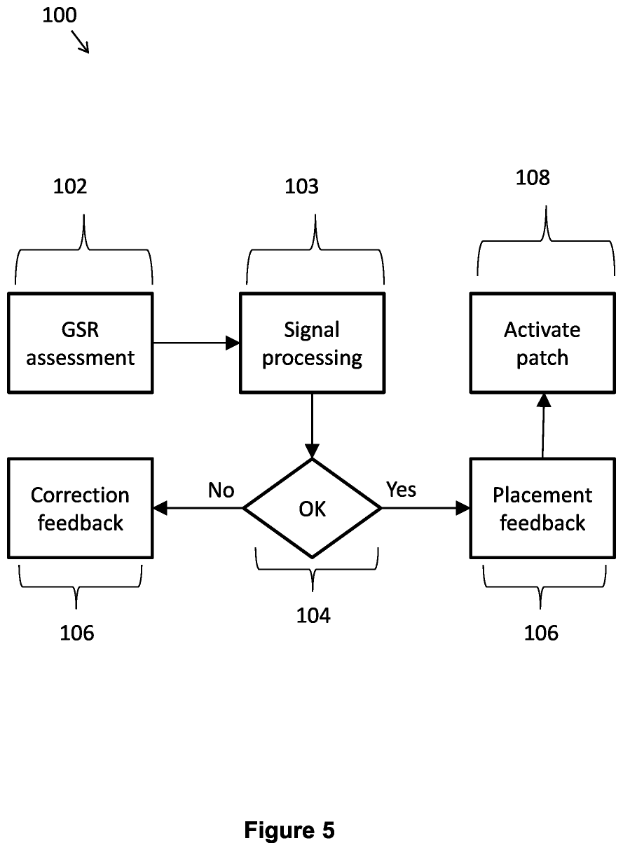

FIG. 5 shows another example of a flowchart of the method 100. The operation 102 is shown as a “GSR assessment” process. At an operation 103, the electronic processor 16 performs a signal processing operation on the skin measurement data 15. If the skin measurement data 15 is determined to not be ok from the signal processing operation 103, then the operation 104 is performed, and advice is output in the operation 106 that the patch 19 should be repositioned (i.e., a “correction feedback” process). If the skin measurement data 15 is determined to be ok from the signal processing operation 103, then the operation 104 is performed, and advice is output in the operation 106 that the patch 11 can be attached to the skin of the patient P (i.e., a “placement feedback” process). The operation 108 then occurs in which the vital sign sensor 12 is attached to the skin and measures the vital sign data (i.e., an “activate patch” process).

In some embodiments, the GSR placement sensor 14 is implemented in a separate device (i.e., the second patch 19). This device 19 can be used before actual attachment of the wearable monitoring patch 10 to check that the skin is well prepared.

The GSR skin measurement data 15 is processed to determine whether skin preparation is sufficient to guarantee that the monitoring patch 10 will be adhesive to the skin for the intended period and will not come off earlier.

In some embodiments, the GSR measurement data 15 is used to predict the expected duration of adhesion given the current measurement, so that the patient P (or physician) can judge whether that is sufficient for the current purpose.

In some embodiments, the monitoring patch 10 can include extra sensors that will help predict the adhesion quality/duration of the patch 10.

In other embodiments (e.g., FIGS. 1 and 2), the GSR sensor 14 is embedded in the monitoring patch 10 itself. In this case the above functionality for checking whether the skin well prepared is still relevant, although it is less easy to take the wearable off again in case it is not. Nevertheless, it is a relevant check before patients start the measurement period in which they are wearing the monitoring patch 10.

When the GSR sensor 14 is embedded in the monitoring patch 10 (e.g., FIG. 1 or FIG. 2), the GSR sensor 14 can check whether the monitoring patch 10 makes sufficient contact with the skin. This measurement can be done after attachment of the monitoring patch 10, but before the measuring of the vital sign data 13 is started. This would still allow to reapply of replace the current patch 11.

The measurement can also be done at regular instances during the wearing period of the monitoring patch 10 to check that the patch 11 is still sufficiently adhesive. If not, the patient is warned to reapply or replace the patch 11. Since the connection measurements are done by GSR, this measurement involves applying a small current/voltage to the skin in order to measure the skin's resistance. The application of this measurement current might interfere with the main measurement of the monitoring patch 10. Therefore, the electronic processor 16 regulates when the GSR-based skin contact measurements 15 are taken, and also regulates that no vital sign measurements 13 are done during the GSR measurements 15 (since they cannot be trusted and might impact processing of the vital sign measurements 13).

In the illustrative embodiments, the skin condition sensor 14 is a GSR sensor 14 which acquires GSR measurements as resistance (V/I) or conductance (I/V) values. However, other types of skin condition sensors are contemplated. For example, a Sebumeter® could be employed in place of the illustrative GSR sensor. A Sebumeter® performs an optical reflectance measurement to detect sebum (oil) on the skin.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A system, comprising:
a monitoring patch configured to be adhesively attached to skin of an associated patient, the monitoring patch including at least one vital sign sensor arranged to measure vital sign data of the associated patient when the monitoring patch is adhesively attached to the skin of the associated patient, the monitoring patch further including a wireless transceiver or transmitter configured to wirelessly transmit the vital sign data;
a monitoring device configured to receive the wirelessly transmitted vital sign data and to store, retransmit, or both store and retransmit the vital sign data;
a skin condition sensor configured to measure skin measurement data on the skin of the associated patient; and
at least one hardware processor configured to:
analyze the skin measurement data to determine advice on adhesive attachment of the monitoring patch to the skin; and
output the advice on the monitoring patch or the monitoring device.

2. The system of claim 1, wherein the advice comprises a recommendation as to whether the skin is properly prepared for adhesive attachment of the monitoring patch to the skin.

3. The system of claim 2, wherein the monitoring patch includes an LED, and the at least one hardware processor is configured to:
output the advice as to whether the skin is properly prepared for adhesive attachment of the monitoring patch to the skin by operating the LED in a first state if the skin is properly prepared and in a second state different from the first state if the skin is not properly prepared.

4. The system of claim 1, wherein the advice comprises an estimated remaining operating time of the monitoring patch adhesively attached to the skin.

5. The system of claim 1, wherein:
the skin condition sensor includes electrodes integrated with the monitoring patch and arranged to electrically contact the skin when the monitoring patch is adhesively attached to the skin of the associated patient.

6. The system of claim 1, wherein at least one vital sign sensor comprises an electrocardiogram or heart rate monitor.

7. The system of claim 1, wherein:
at least one hardware processor includes an on-board hardware processor integral with or configured to be attached to the monitoring patch; and
the skin condition sensor comprises a galvanic skin response (GSR) sensor including GSR electrodes integral with the monitoring patch and arranged to electrically contact the skin when the monitoring patch is adhesively attached to the skin of the associated patient, and an electrical source connected to apply electrical current or voltage to the GSR electrodes, the GSR sensor further including the on-board hardware processor configured to acquire the skin measurement data comprising GSR data using the electrical source and the GSR electrodes.

8. The system of claim 7, wherein:
at least one vital sign sensor includes vital sign electrodes integral with the monitoring patch and arranged to electrically contact the skin when the monitoring patch is adhesively attached to the skin of the associated patient, the at least one vital sign sensor further including the on-board hardware processor configured to acquire the vital sign data using the vital sign electrodes.

9. The system of claim 8, wherein the GSR electrodes are the same set of electrodes as the vital sign electrodes.

10. The system of claim 9, wherein the on-board hardware processor is configured to:
control operation of the vital sign sensor and the GSR sensor to not measure the vital sign data using the vital sign sensor when the GSR sensor is operating to acquire the GSR data.

11. The system of claim 1, wherein at least one sensor includes a galvanic skin response (GSR) sensor configured to measure a condition of the skin of the associated patient on which the monitoring device is disposed.

12. The system of claim 11, wherein the monitoring device includes a vital sign sensor, the electronic processor, and a wireless transmitter or transceiver disposed on a patch attachable to a portion of the associated patient, and the GSR sensor is disposed on a second, separate patch, attachable to a portion of the associated patient.

13. A health status monitoring method, comprising:
measuring, with a skin condition sensor attached to skin of an associated patient, skin measurement data on the skin of the associated patient;
analyzing, with at least one hardware processor, the skin measurement data to determine advice on adhesive attachment of a monitoring patch to the skin; and
outputting the advice.

14. The method of claim 13, wherein the measuring includes:
measuring a status of the monitoring patch attached to the skin of the associated patient.

15. The method of claim 13, further comprising:
measuring, with at least one vital sign sensor disposed on the monitoring patch, vital sign data of the associated patient when the monitoring patch is adhesively attached to the skin of the associated patient.
